(19) United States
(12) Patent Application Publication
(10) Pub. No.: US 2002/0072062 A1
(43) Pub. Date: Jun. 13, 2002

(54) Chloramphenicol biosynthetic pathway and gene cluster

(75) Inventors: Ashish Paradkar, San Diego, CA (US); Jay M. Short, Rancho Santa Fe, CA (US); Eric J. Mathur, Carlsbad, CA (US); Brian D. Green, San Diego, CA (US)

Correspondence Address: GARY CARY WARE & FRIENDENRICH LLP, 4365 EXECUTIVE DRIVE, SUITE 1600, SAN DIEGO, CA, 92121-2189, US

(73) Assignee: Diversa Corporation

(21) Appl. No.: 09/924,207

(22) Filed: Aug. 7, 2001

Related U.S. Application Data
(62) Division of application No. 09/570,220, filed on May 12, 2000.
(60) Provisional application No. 60/134,323, filed on May 14, 1999.

(51) Int. Cl. C12Q 1/68; G01N 33/53; C12N 1/21; C12P 21/02; C12N 15/74

(52) U.S. Cl. 435/6; 435/69.1; 435/7.1; 435/252.3; 435/320.1

(57) ABSTRACT

The present invention provides a chloramphenicol gene cluster and methods of use thereof. Such gene clusters are useful for production of chloramphenicol.

### Background of the Invention

Chloramphenicol is an N-dichloroacyl phenylpropanoid antibiotic produced by Streptomyces venezuelae. Other strains which produce chloramphenicol include Streptomyces pheochromogenes and Streptomyces venezuelae 13S. Corynebacterium hydrocarboclastus makes a related metabolite called corynecin.

Chloramphenicol is a broad spectrum antibiotic, and although it demonstrates some side effects in humans, it is a clinically important drug that is especially effective against typhoid, meningitis, and other microbially related diseases.

Chloramphenicol is synthesized by S. venezuelae as follows:

Chorismic acid→p-aminophenylalanine→p-aminophenylserine→Dichloroacetyl-p-aminophenylserine→Chloramphenicol.

Chloramphenicol biosynthesis genes are located on the chromosome of S. venezuelae ATCC 10712. Mutants blocked in the production of chloramphenicol have been generated in the labs of L. C. Vining and C. Stuttard at Dalhousie University (Doull et al., 1985). The cml mutations present in these mutants have been used to define the organization of cml genes in this organism. Conjugation and transductional analysis has indicated that all cml genes form a tight cluster on the chromosome (FIG. 1, Vats et al. 1987).

### SUMMARY OF THE INVENTION

The present invention provides a chloramphenicol synthesis gene cluster. In one embodiment, the gene cluster is produced by the process comprising: (a) forming a genomic clone library of a chloramphenicol producing microbe; (b) transfecting clones from said library into donor host cells; (c) mating the transfected donor host cells with a chloramphenicol producing microbe, said microbe comprising a mutation in the chloramphenicol gene cluster; (d) screening the resulting recombinant clones for production of chloramphenicol; and (e) isolating said gene cluster from the clones positive for chloramphenicol production. In a preferred embodiment, the donor host cell is E. coli. Preferably, the chloramphenicol producing microbe is S. venezuelae.

Preferably, the mutation in the gene cluster results in the inactivation of a gene encoding a protein involved in the chloramphenicol synthesis pathway, and/or the production of a non-functional protein involved in such pathway.

The present invention further provides a method of producing a clone containing a chloramphenicol gene cluster, the method comprising: (a) forming a genomic clone library of a chloramphenicol producing microbe; (b) transfecting clones from said library into donor host cells; (c) mating the transfected donor host cells with a chloramphenicol producing microbe, said microbe comprising a mutation in the chloramphenicol gene cluster; (d) screening the resulting recombinant clones for production of chloramphenicol; and (e) isolating clones positive for chloramphenicol production.

The present invention also provides a method of producing chloramphenicol comprising: (a) culturing the clone produced according to the method described herein; and (b) isolating the resulting chloramphenicol. In a preferred embodiment, the production is increased relative to the wild-type strain of S. venezuelae.

### BRIEF DESCRIPTION OF THE DRAWINGS

FIG. 1 is a schematic representation of the relative positions of the cml genes within the cml gene cluster.

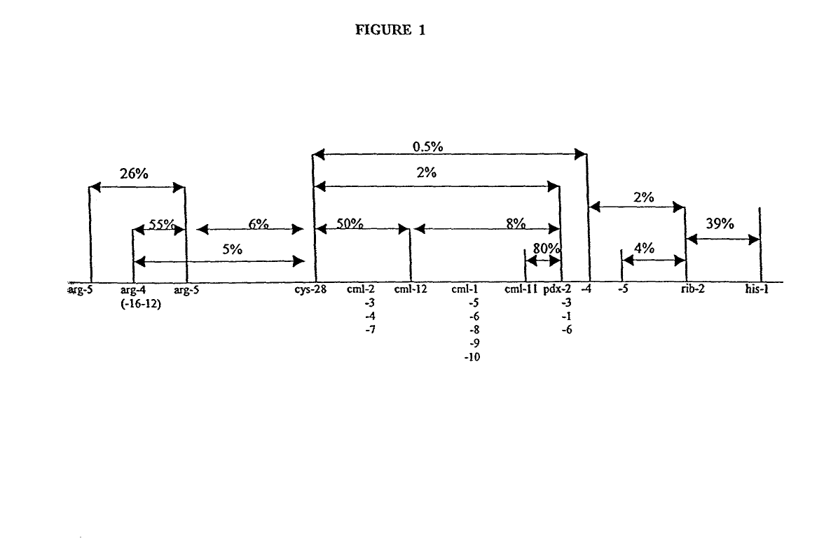

FIG. 2 shows results of exconjugants grown on a bioassay plate containing MYM agar and apramycin.

FIG. 3 shows h.p.l.c. analysis comparing chloramphenicol production.

FIG. 4 shows results of exconjugants grown on a bioassay plate containing MYM agar and apramycin, and bioassayed with Micrococcus luteus.

### DETAILED DESCRIPTION OF THE INVENTION

The strategy used to clone cml genes was to complement cml-5 or cml-12 mutations and to examine for chloramphenicol production.

Materials

VS153 (trpC, cml-5) is a chloramphenicol non-producing strain derived from VS35 by mutagenesis. VS35 is derived from the wild type strain (Stuttard, C., FEMS Microbiol. Lett. 20:467-470 (1983)). It is blocked in a step before p-aminophenylserine and after p-aminophenylalanine intermediates in the pathway.

VS503 (cml-12, pdx-4, hsp-11) is a chloramphenicol non-producing strain that is derived from the wild type strain (Sushma Vats, 1987 Ph.D. Thesis, Dalhousie University, Halifax, N.S., Canada). It is blocked in a step before p-aminophenylalanine formation.

DS154 (trpC, cml-5, jad-1::hygR) is derived from VS153 by introduction of the jad-1::hyg mutation. The knockout was constructed by inserting hygromycin resistance gene (hyg) (Zalacain et al., 1986) within orf1 (jad-1). Thejad1::hyg mutation was introduced in strain VS153 to stop jadomycin production, which is another antibiotic produced by S. venezuelae (See also, Han et at., 1994).

Methods

Cloning of a DNA Fragment Which Complements cml-5, and cml-12 Mutations in VS153, and VS503, Respectively.

a. Construction of a S. venezuelae library.

Genomic DNA of S. venezuelae ATCC10712 was isolated using standard methods (Hopwood et al., 1985, Genetic Manipulations of Streptomyces, a laboratory manual, Norwich, UK, John Innes Foundation), and partially digested with Sau3Al. The cut genomic DNA was size-fractionated on a sucrose gradient and fragments more than 24 kb in size were ligated to the cosmid pOJ446 arms (Bierman et al., 1992). The cosmid arms were prepared by first cutting pOJ446 with HpaI, then treating with shrimp alkaline phosphatase followed by digestion with BamHI. The resulting ligation mixture was packaged with λ-packaging system, and transfected into E. coli STR611. STR611 is a Diversa strain which is used as a donor strain for mating DNA libraries into S. venezuelae. It is derived from strain GM2163 (dam) by incorporating the mutations mcrC-mrr, and introducing the mobilizing plasmid pUZ8002. The DNA in strain STR611 is non-methylated and is thus introduced into Streptomyces venezuelae at high efficiency.

b. Introduction of S. venezuelae genomic library into DS154

The STR611 E. coli containing S. venezuelae library was mated into S. venezuelae DS154 (cml-5) strain by a mating protocol as follows:

100 μl of spores of S. venezuelae were suspended in 0.4 ml of MYM liquid medium (maltose-yeast extract-malt extract) and heat shocked at 50 C. for 10 min. The spores were spun down in a centrifuge and then washed once with 0.4 ml of MYM liquid medium. 0.2 ml of the E. coli library cells were resuspended in 0.5 ml of LB+kan+apr+cml (Luria broth+kanamycin+apramycin+chloramphenicol) and incubated in a shaker for 15 min at 37° C., after which the cells were mixed with the heat treated S. venezuelae spores. The S. venezuelae-E. coli cell mixture was then centrifuged and the pellet washed with 0.5 ml MYM once and the cell mixture was resuspended in 1.1 ml MYM medium. Subsequently, this mixture was spread (0.1 ml per plate) on R2-S+Trp agar (R2-S is similar to R2 medium described in Hopwood et al., 1985, without sucrose; trp is tryptophan). After overnight growth of the mating mixture at room temperature, antibiotics apramycin, nalidixic acid, and chloramphenicol were overlayed to allow only the exconjugants (recombinant clones) to grow.

c. Bioassay of exconjugants for chloramphenicol production

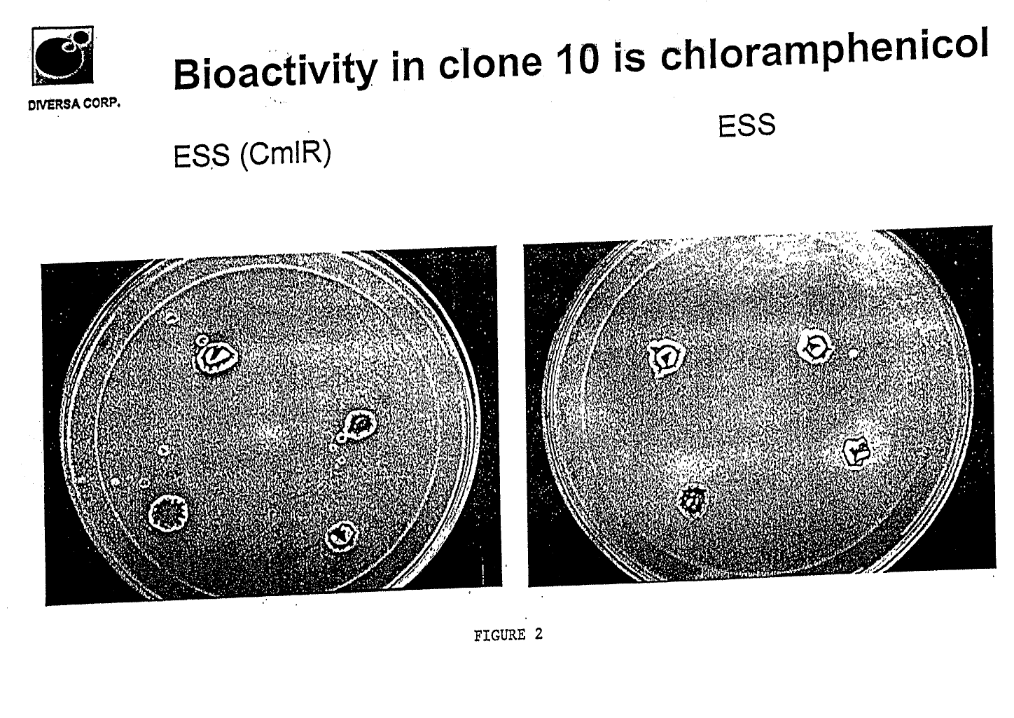

After 3-4 days the exconjugants were picked and patched on a big bioassay plate containing MYM agar+apramycin. After growth of the recombinant clones for 3-4 days at 30° C., the plate containing the clones was overlayed with Micrococcus luteus, a bacterium sensitive to chloramphenicol. After incubation of the plates for 2 days at 30° C., one clone (Clone 10) out of 143 clones showed a big zone of inhibition around it (FIG. 2), while rest of the exconjugants did not show any zone of inhibition. When clone 10 was repatched on MYM+Apr agar medium, grown and bioassayed against M. luteus, the bioactivity was observed again.

d. Analysis to verify that the bioactivity in clone 10 is due to chloramphenicol

The clone 10 was bioassayed against an E. coli ESS strain which is which is chloramphenicol-resistant and an isogenic E. coli ESS strain which is chloramphenicol-sensitive (the original ESS strain was obtained from Dr. Susan Jensen, Univ. of Alberta, Edmonton; Dr. Arnold Demain, MIT, Cambridge, and then modified at Diversa). As shown in FIG. 2, zones of inhibition were seen only in the case of chloramphenicol-sensitive E. coli ESS, but not in the case of chloramphenicol-resistant E. coli ESS suggesting that the bioactivity is due to chloramphenicol.

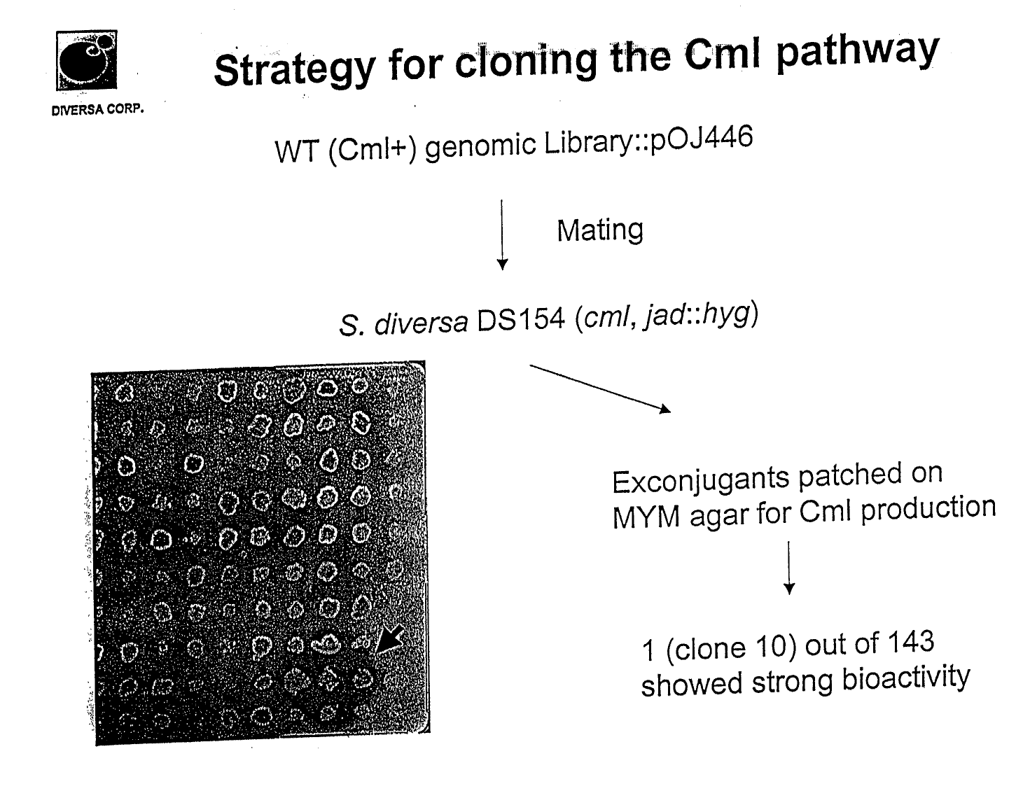

The second evidence that the bioactivity is due to chloramphenicol was obtained by h.p.l.c. analysis. DS154 clone 10 along with S. venezuelae ATCC 10712 (positive control) and VS153 (negative control) strains were grown in MYM liquid culture (apramycin added in the case of DS154 clone 10) for 3-4 days, and the supernatant were extracted using solid phase extraction method. The extracts were resuspended in methanol and subjected to h.p.l.c. analysis. As shown in FIG. 3, DS154-clone 10 showed the peak for chloramphenicol (R.T. 10.7 min) similar to the wild type strain, while the control strain VS153 failed to show any chloramphenicol, as expected.

e. Verification that cml complementing activity is due to the insert on cosmid 10-4

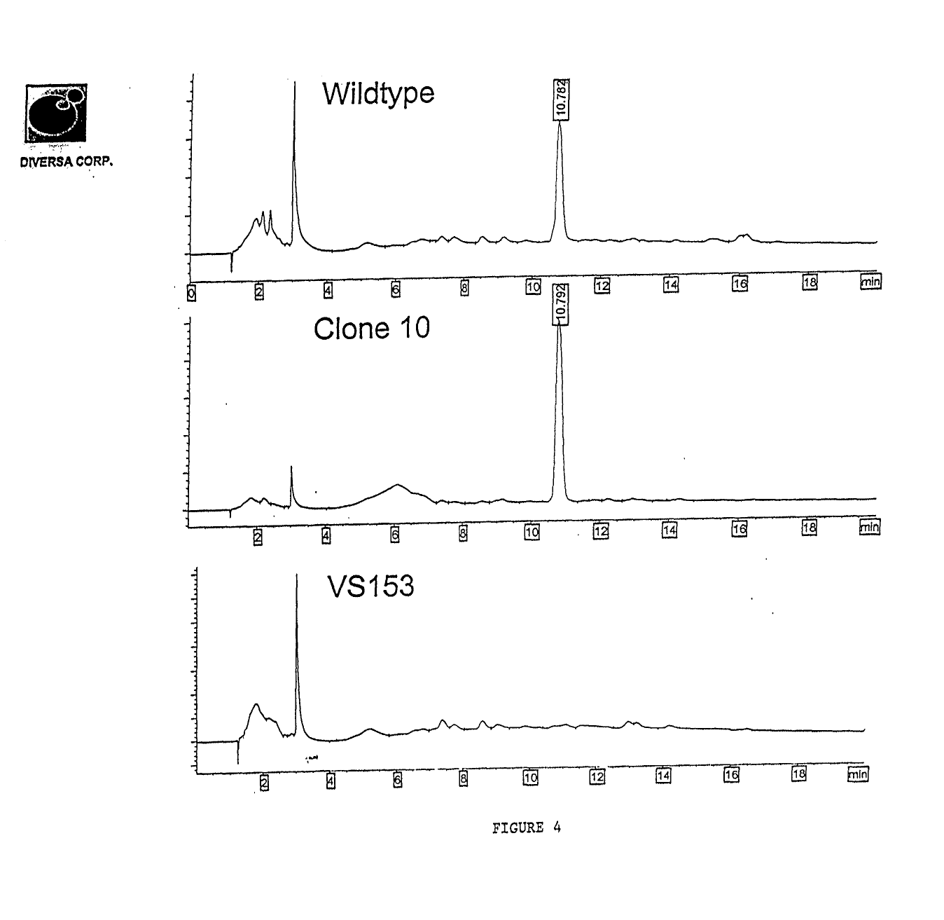

The strain DS154 (clone 10) was grown in MYM liquid medium (plus apramycin) for 48 hours and the mycelia was used to prepare plasmid DNA using methods described in the Streptomyces manual (Hopwood et al., 1985). The resulting plasmid DNA preparation was used to transform electrocompetent DH10B cells. Of several apramycin-resistant colonies obtained, twenty colonies were examined for plasmid content. Four colonies contained plasmid carrying large insert DNA. Out of these one plasmid, pOJ446:10-4 was first introduced into the E. coli donor strain STR611 and from there mated into S. venezuelae DS154 (cml-5) and S. venezuelae VS503 (cml-12). The cosmid pOJ446 (without insert) was also mated from STR611 into S. venezuelae strains DS154 and DS503. Fourteen exconjugants each of DS154 (pOJ446), DS154 (pOJ446:clone 10-4), VS503 (pOJ446) and VS503 (pOJ446:clone 10-4) were patched on MYM agar+apramycin, grown for 4-5 days and bioassayed with M. luteus for detecting chloramphenicol production. As shown in FIG. 4, bioactivity due to chloramphenicol was detected in 13 out of 14 clones of VS503 (pOJ446:clone 10) but not in the case of VS503 containing the vector alone control. This suggests that the cosmid 10-4 contains the cml-12 complementing DNA insert. However, VS154 (clone 10-4) clones failed to show any clear zones of inhibition although hazy zones of inhibition were present that were absent from the vector alone control. It is possible that the original cosmid 10 in the library which complemented cml-5 mutation in strain DS154 underwent deletions or rearrangements during propagation in strain DS154 or underwent deletions or rearrangements in E. coli giving rise to cosmid 10-4 which now can complement cml-12 mutation in strain VS503 but complements cml-5 mutation in strain VS153 only partially.

These results suggest that the cosmid 10 contains DNA fragment that complements cml-12 and cml-5 mutations, and therefore other chloramphenicol biosynthesis genes are very likely to be present on cosmid 10. The cosmid 10-4 is currently being analyzed for DNA sequence.

All references cited herein are incorporated by reference.

References

A role for pabAB, a p-aminobenzoate synthase gene of Streptomyces venezuelae ISP5230, in chloramphenicol biosynthesis.

Microbiology. 1996 June;142 (Pt 6):1345-55.

Brown MP, Aidoo KA, Vining LC.

Department of Biology, Dalhousie University, Halifax, Nova Scotia, Canada.

Mutagenesis of Streptomyces venezuelae ISP5230 and selection for P-aminobenzoic acid-dependent growth in the presence of sulfanilamide yielded Pab mutants (VS519 and VS620) that continued to produce chloramphenicol (Cm), although with increased medium dependence. Transforming the mutants with pDQ102 or pDQ103, which carried a Pab-complementing fragment from S. venezuelae ISP5230 in alternative orientations, restored uniformly high Cm production in VS620, but did not alter the medium dependence of Cm production in VS519. The cloned S. venezuelae DNA fragment was subcloned and trimmed to the minimum size conferring Pab complementation. The resulting 2.8 kb BamH1-Sac1 fragment was sequenced. Codon preference analysis showed one complete ORF encoding a polypeptide of 670 ammo acids. Comparison of the deduced amino acid sequence with database proteins indicated that the N- and C-terminal regions resembled PabA and PabB, respectively, of numerous bacteria. The gene product showed overall sequence similarity to the product of a fused pabAB gene associated with secondary metabolism in Streptomyces griseus. Insertion of an apramycin resistance gene into pabAB cloned in a segregationally unstable vector and replacement of the S. venezuelae chromosomal pabAB with the disrupted copy lowered sulfanilamide resistance from 25 to 5 micrograms mL-1 and blocked Cm production.

Cloning, sequencing and disruption of a bromoperoxidase-catalase gene in Streptomyces venezuelae: evidence that it is not required for chlorination in chloramphenicol biosynthesis.

Microbiology. 1996 March;142 (Pt 3):657-65.

Facey SJ, Gross F, Vining LC, Yang K, van Pee KH

Institut far Mikrobiologie, Universitat Hohenheim, Stuttgart, Germany.

Genomic DNA libraries of Streptomyces venezuelae ISP5230 and of a mutant blocked at the chlorination step of chloramphenicol biosynthesis were probed by hybridization with a synthetic oligonucleotide corresponding to the N-terminal amino acid sequence of a bromoperoxidase-catalase purified from the wild-type strain. Hybridizing fragments obtained from the two strains were cloned and sequenced. Analysis of the nucleotide sequences demonstrated that the fragments contained the same 1449 bp open reading frame with no differences in nucleotide sequence. The deduced polypeptide encoded 483 amino acids with a calculated M(r) of 54,200; the N-terminal sequence was identical to that of the bromoperoxidase-catalase purified from wild-type S. venezuelae. Comparison of the amino acid sequence predicted for the cloned bromoperoxidase-catalase gene (bca) with database protein sequences showed a significant similarity to a group of prokaryotic and eukaryotic catalases, but none to other peroxidases or haloperoxidases. Replacement of the bca gene in the wild-type strain of S. venezuelae with a copy disrupted by insertion of a DNA fragment encoding apramycin resistance did not prevent chloramphenicol production.

Inactivation of chloramphenicol by O-phosphorylation. A novel resistance mechanism in Streptomyces venezuelae ISP5230, a chloramphenicol producer.

J. Biol. Chem. 1995 Nov 10;270(45):27000-6.

Mosher RH, Camp DJ, Yang K, Brown W, Shaw WV, Vining LC.

Biology Department, Dalhousie University. Halifax, Nova Scotia, Canada.

Plasmid pJV4, containing a 2.4-kilobase pair insert of genomic DNA from the chloramphenicol (Cm) producer Streptomyces venezuelae ISP5230, confers resistance when introduced by transformation into the Cm-sensitive host Streptomyces lividans M252 (Mosher, R. H. Ranade, N. P., Schrempf, H., and Vining, L. C. (1990) J. Gen. Microbiol. 136, 293-301). Transformants rapidly metabolized Cm to one major product, which was isolated and purified by reversed phase chromatography. The metabolite was identified by nuclear magnetic resonance spectroscopy and mass spectrometry as 3′-O-phospho-Cm, and was shown to have negligible inhibitory activity against Cm-sensitive Micrococcus luteus. The nucleotide sequence of the S. venezuelae DNA insert in pJV4 contains an open reading frame (ORF) that encodes a polypeptide (19 kDa) with a consensus motif at its NH2 terminus corresponding to a nucleotide-binding amino acid sequence (motif A or P-loop; Walker, J. E., Saraste, M., Runswick, M. J., and Gay, N. J. (1982) EMBO J. 1, 945-951). When a recombinant vector containing this ORF as a 1.6-kilobase pair SmaI-SmaI fragment was used to transform S. lividans M252, uniformly Cm-resistant transformants were obtained. A strain of S. lividans transformed by a vector in which the ORF had been disrupted by an internal deletion yielded clones that were unable to phosphorylate Cm, and exhibited normal susceptibility to the antibiotic.

Chloramphenicol resistance in Streptomyces: cloning and characterization of a chloramphenicol hydrolase gene from Streptomyces venezuelae.

J. Gen. Microbiol. 1990 February;136 (Pt 2):293-301.

Mosher R H, Ranade N P, Schrempf H, Vining L C.

Department of Biology, Dalhousie University, Halifax, Nova Scotia, Canada.

A 6.5 kb DNA fragment containing a chloramphenicol-resistance gene of Streptomyces venezuelae ISP5230 was cloned in Streptomyces lividans M252 using the high-copy-number plasmid vector pIJ702. The gene was located within a 2.4 kb KpnI-SstI fragment of the cloned DNA and encoded an enzyme (chloramphenicol hydrolase) that catalysed removal of the dichloroacetyl moiety from the antibiotic. The deacylated product, p-nitrophenylserinol, was metabolized to p-nitrobenzyl alcohol and other compounds by enzymes present in S. lividans M252. Examination of the genomic DNA from several sources using the cloned 6.5 kb SstI fragment from S. venezuelae ISP5230 as a probe showed a hybridizing region in the DNA from S. venezuelae 13s but none in the DNA from another chloramphenicol producer, Streptomyces phaeochromogenes NRRLB 3559. The resistance phenotype was not expressed when the 6.5 kb SstI fragment or a subfragment was subcloned behind the lac-promoter of plasmid pTZ18R in Escherichia coli.

Transductional analysis of chloramphenicol biosynthesis genes in Streptomyces venezuelae.

J. Bacteriol. 1987 August;169(8):3809-13.

Vats S, Stuttard C, Vining L C

Auxotrophs isolated from two chloramphenicol-nonproducing mutants of Streptomyces venezuelae included three requiring pyridoxal (Px1-), VS248 (cml-11 pdx-2), VS253 (cml-11 pdx-3), and V8258 (cml-12 pdx-4), and one requiring thiosulfate, VS263 (cml-12 cys-28). Results of SV1-mediated transductions were consistent with the relative marker order cys-28-cml-12-cml-11-pdx-2, 3, 4, 5, all of which were cotransducible and must therefore span less than 45 kilobases of DNA, the approximate length of DNA packaged by SV1. cys-28 was also cotransducible with arg-4 and arg-6, but arg and pdx were not cotransducible. Results of crosses with donors carrying any one of 11 cml mutations were consistent with the location of all cml mutations between cys-28 and pdx markers. Also, a new Px1- auxotroph (pdx-6) and two new Cml- mutants were recovered after localized hydroxylamine mutagenesis of a cys-28 cml+strain derived from VS263 by transduction.

PMID: 3475271, UI:87279938

Conjugational fertility and location of chloramphenicol biosynthesis genes on the chromosomal linkage map of Streptomyces venezuelae.

J. Gen. Microbiol. 1986 May;132 (Pt 5):1327-38.

Doull J L, Vats S, Chaliciopoulos M, Stuttard C, Wong K, Vining L C

In Streptomyces venezuelae fertility, defined as chromosomal gene recombination, was enhanced over 1000-fold when one parent in a biparental conjugational cross lacked the physically-undetected plasmid SVP1, as compared with crosses in which both parents carried SVP1. The existence of SVP1 and at least two other fertility plasmids, SVP2 and SVP3, was detected in S. venezuelae by ‘lethal zygosis’ elicited by a plasmid-plus mycelium in contact with a plasmid-minus mycelium. Conjugational crosses were used to construct a linkage map of S. venezuelae which was highly consistent with the map of analogous loci in S. coelicolor A3(2). A cluster of genes governing chloramphenicol biosynthesis was located near arg, cys and pdxB genes at a position rough the 1-2 o'clock region of the S. coelicolor A3(2) map.

Isolation and characterization of Streptomyces venezuelae mutants blocked in chloramphenicol biosynthesis.

J. Gen. Microbiol. 1985 January; 131 (Pt 1):97-104.

Doull J, Ahmed Z, Stuttard C, Vining L C

Twelve Streptomyces venezuelae mutants blocked in chloramphenicol biosynthesis were isolated. Two of these (Cml-1 and Cml-12) were apparently blocked in the conversion of chorismic acid to p-aminophenylalanine and three (Cml-4, Cml-5 and Cml-8) accumulated p-aminophenylalanine and may have been blocked in the hydroxylation reaction that converted this intermediate to p-aminophenylserine. One mutant (Cml-2) accumulated D-threo-1-p-nitrophenyl-2-propionamido-1,3-propanediol and D-threo-1-p-nitrophenyl-2-isobutyramido-1,3-propanediol, indicating that chlorination of the alpha-N-acyl group of chloramphenicol was blocked. The remaining six strains did not excrete any detectable chloramphenicol pathway intermediates.

Evidence for a chromosomal location of the genes coding for chloramphenicol production in Streptomyces venezuelae.

J. Bacteriol. 1983 April; 154(1):239-44.

Ahmed Z U, Vining L C

Of seven chloramphenicol-producing actinomycetes examined, only Streptomyces venezuelae strain 13s contained extrachromosomal DNA detectable by agarose gel electrophoresis and cesium chloride-ethidium bromide density gradient centrifugation. The single 17-megadalton plasmid present in this strain was indistinguishable from plasmid pUC3 previously isolated from mutagenized cultures. Strains selected for their inability to produce chloramphenicol after treatment with acriflavine or ethidium bromide still contained a plasmid that had the same electrophoretic mobility as plasmid pUC3 and yielded similar fragments when digested with restriction endonucleases. By regenerating protoplasts of strain 13s and screening for isolates lacking extrachromosomal DNA, strain PC5 1-5 was obtained. The absence of plasmid pUC3 sequences in this strain was confirmed by Southern hybridization using 32P-labeled plasmid as a probe. Since the plasmidless strain produced as much chloramphenicol as did the parent strain, pUC3 contains neither structural nor regulatory genes for antibiotic production. Evidence from electrophoretic analysis of BamH1 digests of total cellular DNA from wild-type and dye-treated nonproducing progeny indicated that acriflavin caused structural changes in the chromosome.

Refs. Related to the construction of DS154 (jad::hyg strain)

1. Bierman et al. (1992) Plasmid cloning vectors for the conjugal transfer of DNA from Escherichia coli to Streptomcyes spp. Gene 116:43-49.

2. Zalacain et al. (1986) Nucleotide sequence of the hygromycin B phosphotransferase gene from Streptomyces hygroscopicus.

3. Han et al. (1994) Cloning and characterization of polyketide synthase genes for jadomycin b biosynthesis in Streptomyces venezuelae ISP5230. Microbiology 140:3379-3389.

I claim:

1. A method of producing a clone containing a chloramphenicol gene cluster, the method comprising:
(a) forming a genomic clone library of a chloramphenicol producing microbe;
(b) transfecting clones from said library into donor host cells;
(c) mating the transfected donor host cells with a chloramphenicol producing microbe, said microbe comprising a mutation in the chloramphenicol gene cluster;
(d) screening the resulting recombinant clones for production of chloramphenicol; and
(e) isolating clones positive for chloramphenicol production.

2. A method of producing chloramphenicol comprising:
(a) culturing the clone produced according to claim 1; and
(b) isolating the resulting chloramphenicol.

3. A method of identifying a chloramphenicol biosynthesis gene cluster, or analog thereof, in a microbe said method comprising:
(a) isolating a nucleotide sequence from a chloramphenicol biosynthesis gene cluster;
(b) constructing a probe comprising said sequence; and
(c) contacting said probe with a genomic or a DNA library derived from said microbe;
thereby identifying the chloramphenicol biosynthesis gene cluster, or analog thereof.

4. The method of claim 3, wherein said analog is corynecin.

5. The method of claim 3, wherein said nucleotide sequence is derived from a chloramphenicol biosynthesis gene cluster from S. venezuelae.

6. A method of knocking-out genes contained in a chloramphenicol biosynthesis gene cluster, said method comprising:
(a) isolating a nucleotide sequence from a chloramphenicol biosynthesis gene cluster;
(b) introducing a mutation into said nucleotide sequence; and
(c) contacting resulting nucleotide sequence with the chloramphenicol biosynthesis gene cluster of a microbe; said nucleotide sequence homologously recombining with said gene cluster; thereby knocking-out genes contained in said cluster.

7. The method of claim 6, wherein said mutation is a deletion, substitution or insertion of one or more nucleotides.

8. Method of claim 6, wherein said nucleotide sequence is derived from the chloramphenicol biosynthesis gene cluster from S. venezuelae.

9. The expression product of a gene cluster produced by the method of claim 6.
